(19) United States
(12) Patent Application Publication
(10) Pub. No.: US 2005/0010235 A1
(43) Pub. Date: Jan. 13, 2005

(54) SURGICAL DEVICE

(76) Inventor: Gregg A. VanDusseldorp, Crown Point, IN (US)

Correspondence Address: HARTMAN & HARTMAN, P.C., 552 EAST 700 NORTH, VALPARAISO, IN, 46383, US

(21) Appl. No.: 10/604,297

(22) Filed: Jul. 9, 2003

(51) Int. Cl. A61B 17/24

(52) U.S. Cl. 606/113

(57) ABSTRACT

A surgical device having a sheath with an interior passage, legs that project from the passage of the sheath, and actuating means for deploying the legs from the sheath and for retracting the legs into the sheath. The legs are adapted to move outwardly away from each other when deployed from the sheath by the actuating means to establish a deployed position, and to move inwardly toward each other when retracted into the sheath by the actuating means. At least one of the legs has a transverse cross-sectional shape defined by a first surface that is concave and an oppositely-disposed second surface that is convex. The distal ends of the legs may be connected together so that the legs define a basket in the deployed position, and so that the basket collapses as the legs are retracted into the sheath to perform a grasping operation.

### BACKGROUND OF INVENTION

1. Field of the Invention

The present invention generally relates to surgical devices adapted to capture an object within a cavity of the human body, such as when moving, manipulating and extracting biological material during a medical procedure. More particularly, this invention relates to a surgical device comprising elongate members that, when collapsed toward each other, are capable of capturing an object, and wherein at least one of the elongate members has a cross-sectional shape that promotes the ability of the elongate members to expand and collapse relative to the other members.

2. Description of the Related Art

Various instruments are known in the art for surgically removing stones, calculi and other hard materials from the body. An example is an extraction instrument disclosed in U.S. Pat. No. 5,281,230 to Heidmueller as comprising a pair of bowls that are pivoted toward and away from each other by engaging their proximal ends with a sheath. Other types of extraction instruments make use of multiple wires that are flexed to grasp an object. For example, U.S. Pat. No. 5,944,728 to Bates discloses an instrument having arcuate wires with rectangular, round, D-shaped, or V-shaped cross-sections. The wires form a basket when a plunger associated with the instrument is in a distal position, allowing the legs to radially collapse toward each other. To expand the legs, the plunger must be actuated into engagement with the legs, forcing the legs radially apart from each other. As such, surgically moving, manipulating and extracting material from a body cavity is complicated by the requirement to additionally operate the plunger to expand and contract the legs.

Another example of an extraction instrument is disclosed in U.S. Pat. No. 6,203,552 to Bagley et al. As with Bates, the instrument taught by Bagley et al. has arcuate legs that form a collapsible basket when actuated with respect to a sheath. Each leg has a wedge-shaped cross-sectional shape, so that together they fill the cross-sectional area of the sheath. Contrary to Bates, the instrument disclosed by Bagley et al. does not require a separate plunger to expand (dilate) and collapse the basket.

There is an ongoing effort to devise surgical extraction instruments with greater dilating force when expanding to acquire an object, with greater grasping strength when capturing onto an object, and that maintain their form and alignment throughout their range of motion so as to more easily capture biological material during a variety of medical procedures.

### SUMMARY OF INVENTION

The present invention provides a surgical device having a sheath with an interior passage, legs that project from the passage of the sheath, and actuating means for deploying the legs from the sheath and for retracting the legs into the sheath. The legs are adapted to move outwardly away from each other when deployed from the sheath by the actuating means to establish a deployed position, and to move inwardly toward each other when retracted into the sheath by the actuating means. At least one of the legs has a transverse cross-sectional shape defined by a first surface that is concave and an oppositely-disposed second surface that is convex.

According to one aspect of the invention, the legs have adjacent distal ends that are connected together so that the legs define a basket in the deployed position, and so that the basket collapses as the legs are retracted into the sheath by the actuating means so as to define a grasping position in which the legs are operable to perform a grasping operation. According to another aspect of the invention, the cross-sectional shapes of the legs cause the legs to automatically deploy radially outward and away from each other when deployed outside the sheath with the actuating means, such that a plunger is not required to operate the legs. The cross-sectional shape of the legs is capable of contributing greater strength and rigidity to the legs to promote their ability to expand with a sufficient force that eliminates the need for a plunger, to provide greater grasping strength when collapsed, and to maintain their form and alignment throughout their range of motion. As a result, the device is capable of reliably moving, manipulating and extracting biological material in a variety of medical procedures.

Other objects and advantages of this invention will be better appreciated from the following detailed description.

### BRIEF DESCRIPTION OF DRAWINGS

FIGS. 1 and 2 are side and end views, respectively, of a surgical device in a deployed position in accordance with an embodiment of this invention.

FIGS. 3 and 4 are side views of the surgical device of FIG. 1 in intermediate and stowed positions, respectively.

FIG. 5 is a cross-section along line 5B5 of FIG. 1.

FIG. 6 shows a surgical device in accordance with a second embodiment of this invention.

### DETAILED DESCRIPTION

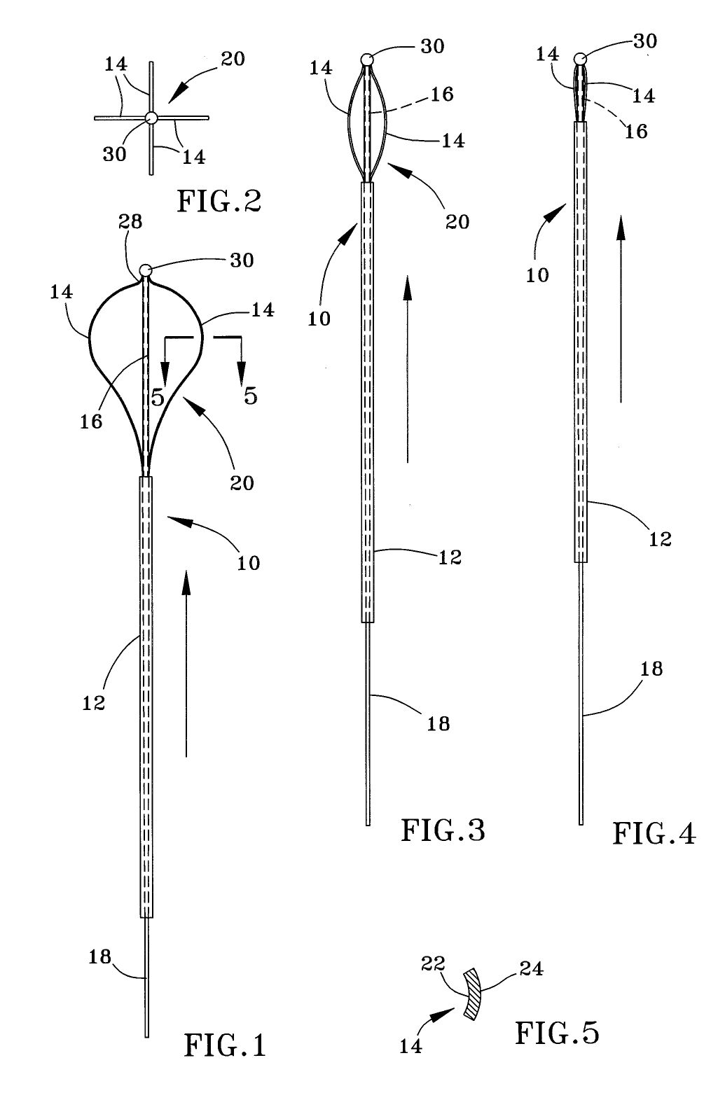

With reference to FIGS. 1 through 4, a surgical device 10 is shown in accordance with a first embodiment of this invention. The device 10 is particularly intended and suitable for extraction procedures, in which biological materials are required to be surgically moved, manipulated and/or extracted from the human body. As such, the device 10 can be adapted for use as, for example, a urological, gynecological, cardiological, laparoscopical or gastro-intestinal instrument.

The extraction device 10 is depicted as comprising a sheath 12, legs 14 that project from a passage within the sheath 12, and a cable 18 (or other suitable actuating member) for simultaneously retracting the legs 14 into the sheath 12 and deploying the legs 14 from the sheath 12. A distal portion 16 of the cable 18 projects from the sheath 12 so as to be surrounded by the legs 14, as seen in FIGS. 1, 3 and 4. The sheath 12 can be formed of any suitable material known in the art. The passage within the sheath 12 can be sized to be sufficiently large to not only accommodate the legs 14 and cable 18, but also provide an irrigation or injection lumen, or a channel for a laser fiber to be passed through so that stones and other biological materials can be captured, held and fragmented to allow the resulting fragments to pass. The sheath 12 may also be equipped with a hollow channel (not shown) through which a sparking wire can be passed to enable the legs 14 (if formed of a conductive material) to be energized with electrosurgical cutting or coagulating current.

In contrast to commonly-assigned U.S. Pat. No. 6,416,519, the embodiment of FIGS. 1 through 4 shows the ends 28 of the legs 14 as being connected together, so that the legs 14 in combination form a basket 20. In FIGS. 1 through 4, the ends 28 of the legs 14 and the distal portion 16 of the cable 18 are interconnected with a distal connector 30 which, depending on the materials used to form the legs 14 and cable 18, can be attached by metallurgical joining (e.g., soldering) or with a mechanical crimp joint. While the device 10 is shown as being equipped with four legs 14, it is foreseeable that fewer or greater numbers of legs could be employed. For example, the device could have two legs 14 (forming, in effect, a snare) or as many as eight legs 14 or more. As represented in FIG. 1, the legs 14 are formed to have a parabolic curved shape, as by such known methods as stamping, rolling, extruding, etc. The legs 14 are formed from a sufficiently rigid material, such as a stainless steel, or a “shape memory” nickel-titanium alloy such as NITINOL, so that the midportions of the legs 14 automatically deploy radially outward and away from each other (and away from the distal portion 16 of the cable 18) when the legs 14 are deployed outside the sheath 12 with the cable 18. As a result, the device 10 does not require a plunger capable of being actuated relative to the legs 14 in order to force the legs 14 radially apart to form the basket 20 of FIG. 1. The legs 14 are sufficiently elastically deformable so that, as the legs 14 are retracted into the sheath 12, the legs 14 elastically collapse radially toward each other to acquire an intermediate position (FIG. 3) in which the basket 20 is partially collapsed. On further retraction into the sheath 12 (FIG. 4), the legs 14 are largely stowed within the sheath 12 and substantially parallel to each other and to the distal portion 16 of the cable 18.

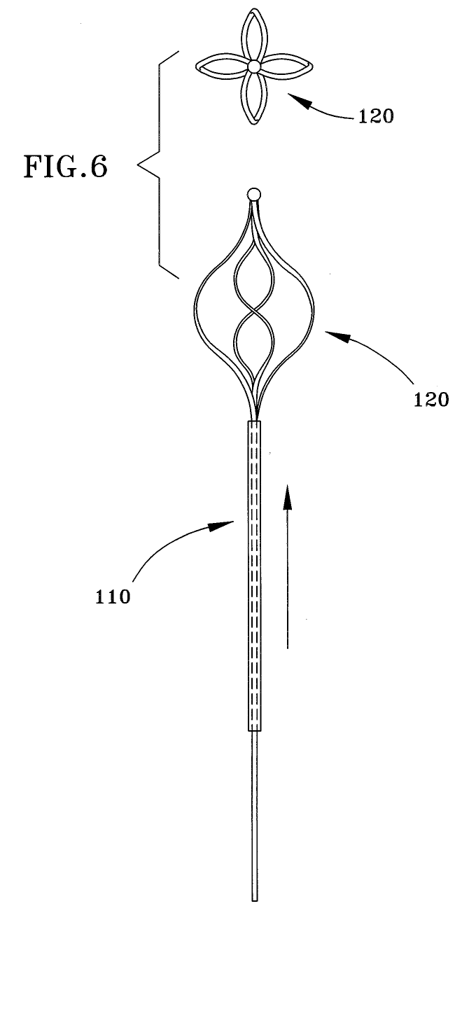

A key feature of the present invention is that each leg 14 has a concave-convex cross-section that contributes greater strength to the legs 14, such that the legs 14 maintain their form and alignment and provide greater grasping strength and expansion force than extraction devices equipped with wires having cross-sectional shapes of the types disclosed in the prior art. As a result, the device 10 is well suited for moving, manipulating and extracting biological material, such as calculi, stones, etc. As depicted in FIG. 5, the legs 14 have a concave-convex cross-section in the sense that the inward surfaces 22 of the legs 14 facing each other are concave, while the oppositely-disposed outward surfaces 24 of the legs 14 are convex. Each surface 22 and 24 preferably has a constant radius of curvature, and the thicknesses of the legs 14 in the direction of a radial of the curvature are substantially constant. The lateral surfaces of the legs 14 are contiguous with the surfaces 22 and 24, and can be of any suitable shape, e.g., rounded, flat such as the radials of the curvatures of the surfaces 22 and 24, etc. When fully collapsed, the legs 14 define a tubular shape in the sense that the legs 14 in combination define a circular exterior cross-section and a circular opening that is sized to accommodate the cable 18.

FIG. 6 shows a second embodiment of an extraction device 110 of this invention, in which the legs 14 have a longitudinal configuration that causes the basket 120 to have a helical shape. Other than the helical shape of the basket 120, the device 110 can have an identical construction to the device 10 of FIGS. 1, 3 and 4.

When used to remove a stone (or calculi or other object) from a cavity of the human body, the legs 14 are extended from the sheath 12 with the cable 18 such that the legs 14 resiliently expand outward to reacquire their parabolic curved shape. Once the stone is surrounded by the legs 14 so as to be nested with the basket 20, the cable 18 is actuated relative to the sheath 12 to retract the legs 14, causing the basket 20 to collapse and grasp the stone. By subsequently extending the legs 14 from the sheath 12, the stone can be released. As such, surgically moving, manipulating and extracting bodies and materials within the human body is performed without additionally operating a plunger or other extraneous component to expand and contract the basket 20 formed by the legs 14.

While the invention has been described in terms of a preferred embodiment, it is apparent that other forms could be adopted by one skilled in the art. For example, appropriate materials could be substituted for those noted. Accordingly, the scope of the invention is to be limited only by the following claims.

1. A surgical device having a sheath with an interior passage, legs that project from the passage of the sheath, and actuating means for deploying the legs from the sheath and for retracting the legs into the sheath, the legs moving outwardly away from each other when deployed from the sheath by the actuating means to establish a deployed position, the legs moving inwardly toward each other when retracted into the sheath by the actuating means, at least one of the legs having a transverse cross-sectional shape defined by a first surface that is concave and an oppositely-disposed second surface that is convex.

2. The surgical device according to claim 1, wherein as a result of their cross-sectional shape, the legs automatically move radially outward and away from each other when deployed outside the sheath with the actuating means without the assistance of a second component to engage and force the legs away from each other.

3. The surgical device according to claim 1, wherein the legs have adjacent distal ends that are connected together so that the legs define a basket in the deployed position and the basket collapses as the legs are retracted into the sheath by the actuating means so as to define a grasping position in which the legs are operable to perform a grasping operation.

4. The surgical device according to claim 3, wherein the legs have a longitudinal shape so that the basket is helical in shape.

5. The surgical device according to claim 1, wherein the legs are capable of being retracted into the sheath with the actuating means to define a stowed position in which the legs are substantially parallel to each other.

6. The surgical device according to claim 5, wherein when in the stowed position the legs define a tubular shape having a circular opening defined by the first surfaces of the legs and a circular exterior cross-section defined by the second surfaces of the legs.

7. The surgical device according to claim 1, wherein each of the legs has the cross-sectional shape.

8. The surgical device according to claim 1, wherein the device has two of the legs and the first surfaces of the two legs face each other.

9. The surgical device according to claim 1, wherein the device has more than two of the legs and the first surfaces of the legs face each other.

10. The surgical device according to claim 1, wherein the device is a surgical instrument chosen from the group consisting of urological, gynecological, cardiological, laparoscopical and gastrointestinal instruments.

11. A surgical extraction device having a sheath with an interior passage, at least three legs that project from the passage of the sheath, and actuating means for deploying the legs from the sheath and for retracting the legs into the sheath, each of the legs comprising:
a transverse cross-sectional shape defined by a concave surface, a convex surface oppositely disposed from the concave surface, and lateral surfaces oppositely disposed from each other; and
a distal end;
wherein the concave surfaces of the legs face each other, the distal ends of the legs are connected together, the legs move outwardly away from each other when deployed from the sheath by the actuating means to establish a deployed position in which the legs define an expanded basket, and the legs are forced to move inwardly toward each other when retracted into the sheath by the actuating means to establish a grasping position in which the legs define a collapsed basket and cooperate to perform a grasping operation.

12. The surgical extraction device according to claim 11, wherein as a result of their cross-sectional shape the legs automatically deploy radially outward and away from each other when deployed outside the sheath with the actuating means without the assistance of a second component to engage and force the legs away from each other.

13. The surgical extraction device according to claim 11, wherein the legs are capable of being retracted into the sheath with the actuating means to define a stowed position in which the legs are substantially parallel to each other.

14. The surgical extraction device according to claim 13, wherein when in the stowed position the legs define a tubular shape having a circular exterior cross-section defined by the convex surfaces of the legs and a circular opening defined by the concave surfaces of the legs.

15. The surgical extraction device according to claim 11, wherein the legs have a longitudinal shape so that the expanded basket is helical in shape.

16. The surgical extraction device according to claim 11, wherein the device is a urological instrument.

17. The surgical extraction device according to claim 11, wherein the device is a gynecological instrument.

18. The surgical extraction device according to claim 11, wherein the device is a cardiological instrument.

19. The surgical extraction device according to claim 11, wherein the device is a laparoscopical instrument.

20. The surgical extraction device according to claim 11, wherein the device is a gastrointestinal instrument.
